(19) United States
(12) Patent Application Publication
(10) Pub. No.: US 2011/0076598 A1
(43) Pub. Date: Mar. 31, 2011

(54) CORRIN COMPOUND FUEL CELL CATALYSTS

(75) Inventors: Kuei-Hsien CHEN, Taipei (TW); Chen-Hao WANG, Sindian City (TW); Hsin-Cheng HSU, Pitou Township (TW); Sun-Tang CHANG, Taoyuan City (TW); Li-Chyong CHEN, Taipei (TW)

(73) Assignee: ACADEMIA SINICA, Taipei (TW)

(21) Appl. No.: 12/570,867

(22) Filed: Sep. 30, 2009

(51) Int. Cl. H01M 4/88; C07D 487/22; H01M 4/00; B01J 31/12; C07H 23/00

(52) U.S. Cl. 429/527; 540/145; 502/152; 502/101; 536/26.44

(57) ABSTRACT

The invention provides metal-containing corrin compounds as catalysts for oxygen reduction in electrochemical devices, such as in fuel cells. The catalysts provide more efficient reduction at lower cost than conventional noble metal catalyst. Methods for preparing the catalysts are also provided.

### BACKGROUND OF THE INVENTION

Energy generation is a very important environmental issue in the 21^{st} century. Conventional energy generators are internal combustion engines, which generate energy by consuming fossil fuels. The efficiency of internal combustion engines is low, and such engines emit undesirable greenhouse gases such as CO, CO₂, SOₓ, and NO_{R}. Therefore, clean and efficient energy generation is desirable.

Fuel cells are electrochemical devices that continuously convert hydrogen or hydrocarbons (fuel) and oxygen (oxidant) into CO₂ and water and output electricity, and are considered to be efficient and eco-friendly energy generators. For example, a proton exchange membrane fuel cell, such as a hydrogen fuel cell, contains a membrane electrode assembly (MEA). An MEA typically contains a solid electrolyte, which is comprised of solid ionomer or ion-exchange membrane sandwiched between two planar electrodes, an anode, and a cathode. The electrodes are composed of catalyst layers deposited on porous sheets which are electrically conductive material, e.g., carbon cloth or carbon paper. The catalyst layers are usually composed of highly reactive and finely comminuted catalysts, such as Pt catalysts, deposited on highly conductive and porous activated carbon.

The oxygen reduction reaction that occurs in the cathode is much more sluggish than the hydrogen oxidation reaction that occurs in the anode. In aqueous acid solution, Pt catalysts are at least 10⁶ times more active for hydrogen oxidation than for oxygen reduction. The oxygen reduction reaction in aqueous media is governed by a number of possible reactions. Two of the most likely reactions that proceed in aqueous acid are:

4 electron-pathway transfer: O₂+4H⁺+4e⁻→2H₂O

2 electron-pathway transfer: O₂+2H⁺+2e⁻→H₂O₂

The 4 electron-pathway transfer is the most attractive for oxygen reduction because it provides the highest cell voltage for the fuel cells. Noble metals such as Pt, Pd, Au, Rh, and their alloys are typically selected as the catalysts of choice for oxygen reduction. To date, fuel cells are far from commercialization due to their high cost. The use of non-noble metal catalysts in the cathode catalyst layer is an attractive method, and is one of the key technologies being investigated for reducing the costs of fuel cells.

In attempts to discover alternatives to noble metal catalysts for fuel cells, some research has focused on transition metal macrocycles. For example, in 1964, Jasinski reported that cobalt phthalocyanine adsorbed on carbon and nickel electrodes acted as a promising catalyst for oxygen reduction (Jasinski, Nature, 201, 1212-1213 (1964)). After this work, many other transition metal macrocycles, e.g., N₄-complexes, were reported, including porphyrin (see Mocchi and Trasatti, Journal of Molecular Catalysis A: Chemical, 204-205, 713-720 (2003); Bogdanoff, Herrmann et al., Journal of New Materials for Electrochemical Systems, 7, 85-92 (2004); Liu, Song et al., Electrochimica Acta, 52, 4532-4538 (2007); Savastenko, Bruser et al., Journal of Power Sources, 165, 24-33 (2007); Xie, Ma et al., Electrochimica Acta, 52, 2091-2096 (2007)), phthalocyanine (see Lu and Reddy, Electrochimica Acta, 52, 2562-2569 (2007); Jingjie, Haolin et al., Electrochimica Acta, 54, 1473-1477 (2009)), and tetraazannulene (see Convert, Coutanceau et al., Journal of Applied Electrochemistry, 31, 945-952 (2001)). Such macrocycles contained transition metals including Mn, Ru, Pd, Pt, Ir, Cr, Ni, Cu, Zn, Mo, Al, Sn, Sb, Ga, etc. Transition metal macrocycles as catalysts have also been described. (See, for example, U.S. Pat. Nos. 3,410,727; 3,617,388; 3,821,028; 3,930,884; 5,683,829; and 6,245,707.)

Beck (Journal of Applied Electrochemistry, 7, 239-245 (1977)) and Zagal (Coordination Chemistry Reviews, 119, 89-136 (1992)) proposed that the oxygen reduction mechanisms of N₄-complexes (XMe) were based on the following steps:

Step 1: XMe^{II}+O₂ [symbol] (XMe^{δ+} . . . O₂^{δ-})

Step 2: (XMe^{δ+} . . . O₂^{δ-})+H⁺→(XMe^{III} . . . O₂H)⁺

Step 3: (XMe^{III} . . . O₂H)⁺+H⁺+2e⁻→XMe+H₂O₂

Step 4: H₂O₂→H₂O+½O₂ (chemical reaction)

It can be seen that Step 3 is a two-electron pathway. Bouwkamp-Wijnoltz et al. (Electrochimica Acta, 43(21-22), 3141-3152 (1998)) used CIFeTMPP (Fe—N₄ materials) as catalysts for oxygen reduction. They concluded that the desired four-electron pathway plays a dominant role only at high potential with low current densities, and that the production of hydrogen peroxide dominates at low potential with high current densities. For fuel cell application, high current densities are usually employed by the practical system, and thus transition metal macrocycles follow the low efficiency two-electron pathway rather than the four-electron pathway

However, the long-term stability of these complexes is often problematic due to the deterioration of the catalysts by the hydrogen peroxide produced. Accordingly, efficient, highly stable, low cost catalysts would be desirable.

### BRIEF SUMMARY OF THE INVENTION

According to an embodiment of the present invention, a fuel cell cathode catalyst comprises at least one metal-containing corrin compound.

Also according to an embodiment of the present invention, a method for forming a fuel cell cathode catalyst comprises depositing at least one metal-containing corrin compound onto a conductive support.

According to a further embodiment of the present invention, a fuel cell comprises a cathode catalyst comprising at least one metal-containing corrin compound.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 is a graph of linear sweep voltammetries of a vitamin B12-derived catalyst according to an embodiment of the present invention; and

FIG. 2 is a graph of lifetime cycles of a vitamin B12-derived catalyst according to an embodiment of the present invention.

### DETAILED DESCRIPTION OF THE INVENTION

The present invention relates to catalysts for oxygen reduction in electrochemical devices, such as fuel cell cathode catalysts. The catalysts comprise corrin rings in non-noble metal-containing corrin structures, and exhibit excellent electrochemical activity. The corrin-containing catalysts are much less expensive than noble-metal catalysts and exhibit better stability than previously known transition metal macrocycles, e.g., N₄ complexes.

As shown in formula (1), a corrin ring contains 4 pyrrole subunits that are joined on opposite sides by a C—CH₃ methylene link, on one side by a C—H methylene link, and with two of the pyrroles joined directly.

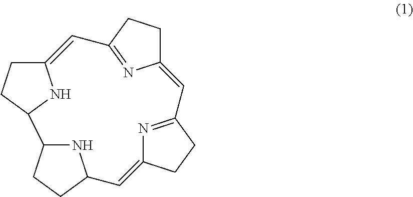

When the center of a corrin ring contains a transition metal which is linked to four reduced pyrrole rings, it forms a corrin-structure macrocycle. For the purposes of this disclosure, the terms “corrin,” “corrin ring,” and “corrin structure” may be understood to be synonymous. As used herein, “corrin compound” and “corrin-containing compound” refer to a compound that contains corrin or a derivative of corrin.

For example, vitamin B12 is a corrin-containing compound which comprises a cobalt-containing corrin ring, as depicted in formula (2).

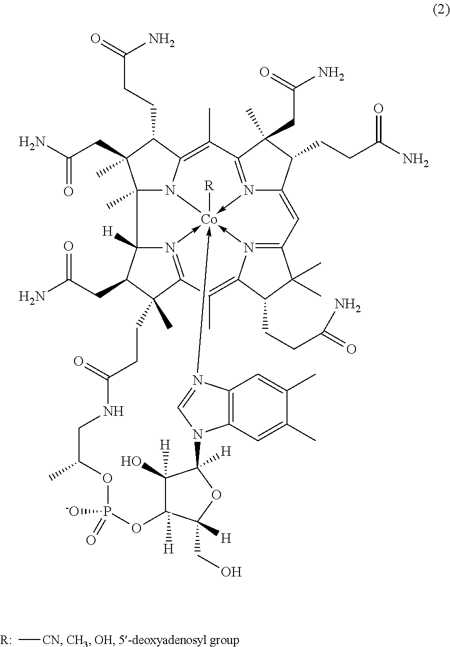

It can be seen in formula (2) that the structure of B12 is based on a corrin ring, in which the central metal ion is cobalt. There are four forms of B12. In all of the forms, four of the six coordination sites are provided by the corrin ring, and a fifth by a dimethylbenzimidazole group. The sixth coordination site, the center of reactivity, “R,” is variable, and may be a cyano group (—CN), a hydroxyl group (—OH), a methyl group (—CH₃) or a 5′-deoxyadenosyl group (in which the C5′ atom of the deoxyribose forms the covalent bond with Co). When R is a cyano group, the compound is called cyano-B12 or cyanocobalamin.

The catalysts according to embodiments of the present invention for oxygen reduction in electrochemical devices, such as in fuel cells, may also be referred to as “fuel cell cathode catalysts.” Such catalysts comprise at least one metal-containing corrin compound, such as a corrin structure macrocycle in one embodiment (when the metal is a transition metal linked to the four reduced pyrrole rings in corrin). In a preferred embodiment, the metal-containing corrin compound comprises one or more metal-containing corrin rings, as shown in formulas (3a) to (3d) below, as the active sites to catalyze oxygen reduction in the fuel cell. As shown in formulas (3c) and (3d), one of the outer ligands may be connected to the metal center of the corrin ring. It may be understood that the metal-containing corrin ring compounds shown in formulas (3a) to (3d) may serve as the fuel cell catalysts. Alternatively, such structures may be part of a larger compound and merely act as the active site, as in the vitamin B12-derived catalyst described below.

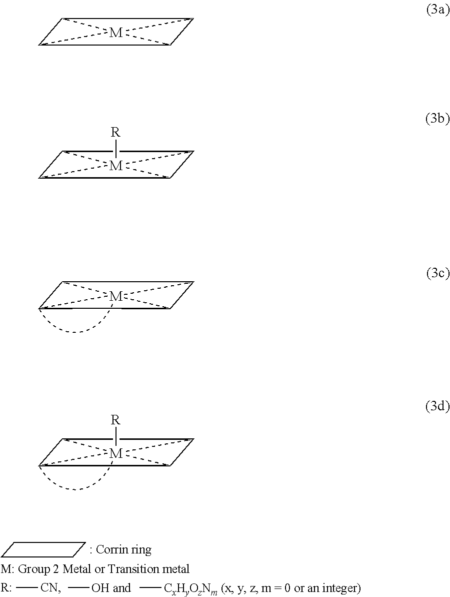

In formulas (3a) to (3d), R is selected from the group consisting of CN, OH, and CₓH_{y}O_{z}Nₘ (x, y, z, and m may be 0 or any integer) and M is a Group 2 metal or a transition metal, preferably a transition metal selected from Groups 5 to 12 of the periodic table. In a more preferred embodiment, the metal-containing corrin compound(s) comprise a metal selected from Fe, Co, and Ni because these metals are particularly active for oxygen reduction. The metal may be in a reduced state (such as Co⁰) or an oxidized state (such as Co¹⁺, Co²⁺ and Co³⁺), and the valence of the metal may change while catalyzing the oxygen reduction.

Metal-containing corrin compounds are well known, including naturally occurring products (such as Vitamin B12, described above) and synthetic compounds. In addition to cobalt-containing corrin compounds, corrin compounds containing various Group 2 metals and transition metals, such as nickel and iron, for example, have also been described in the literature, such as in U.S. Pat. No. 5,345,032. Fuel cell cathode catalysts according to the present invention may comprise both known and novel metal-containing corrin compounds, such as the vitamin B12-derived catalyst described below. It has been found that insoluble metal-containing corrin compounds, such as vitamin B12-derivatives and compounds having formula (3), have high activity for catalyzing oxygen reduction.

A preferred fuel cell cathode catalyst according to the invention is a catalyst derived from vitamin B12. In order to provide sufficient catalytic activity for oxygen reduction, it is necessary to pyrolyze vitamin B12 to prepare an effective catalyst. Additionally, because fuel cells need water to maintain the ionic conductivity of the electrolyte, a vitamin B12-derived catalyst without pyrolysis is not suitable in a fuel cell because it is water soluble. However, after pyrolysis under high temperature treatment (such as about 500 to 900° C.), the outer ligands and the hydrophilic groups of vitamin B12 are removed and neutralized, and the remaining metal-containing corrin compound has high catalytic activity. Therefore, vitamin B12 may be considered to be a precursor for fabricating a preferred metal-containing corrin compound catalyst according to the present invention. However, an identical corrin compound may also be synthesized directly for preparation of the catalyst.

The catalyst material according to the invention may additionally comprise at least one conductive support, such as a carbon- or metal-based material. Exemplary conductive supports include, without limitation, carbon black, carbon fibers, carbon nanotubes, gold particles, etc. The conductive supports not only act as electrical conductors to enhance electrical conductivity but also stabilize the catalyst for oxygen reduction. The metal-containing corrin compound(s) may be deposited on the conductive support by any method know in the art or to be discovered. For example, the corrin compound catalyst material and carbon black may be stirred in a water solution and filtered to provide a catalyst/carbon black slurry in which the catalyst is adsorbed onto the carbon black. The resulting slurry may then be pyrolyzed (such as in an inert atmosphere at high temperature (such as about 600° C.)) to produce chemical bonds between the catalyst and the carbon black. The resulting material would then be referred to as “a catalyst deposited on a conductive support.”

The catalyst material may also be covalently attached to the conductive support using a coupling agent that is bonded to surface functional groups of the support. For example, functional groups such as —COH and —COOH on carbon black surfaces are beneficial for enhancing adsorption between corrin-containing catalysts and the carbon black. Such groups may be produced by subjecting carbon black, for example, to high-temperature treatment in an oxygen-rich atmosphere. The resulting —COH and —COOH groups on the carbon black then bond to the carbonyl groups in vitamin B12-derived catalysts, for example, upon heat treatment of a catalyst/carbon black slurry, as described above. However, other functional groups would also be appropriate for acting as coupling agents to the surface functional groups of the support.

In one embodiment, the metal-containing corrin compound comprises a metal complex derived from an organic or polymeric compound, such as vitamin-B12, functioning as a precursor. The metal-containing corrin compound may be directly chemisorbed from a solution of the compound onto the conductive support as described above, for example. As previously explained, the organic or polymeric compound itself, such as vitamin B12, may not exhibit catalytic activity and/or may not be suitable for catalyzing oxygen reduction and application to a fuel cell. However, after pyrolysis, for example, which destroys the outer ligands of the compound, the metal-containing corrin compound exhibits the desired catalytic activity. The pyrolysis also provides stability to the catalyst.

The catalyst materials according to the present invention comprising at least one metal-containing corrin compound may further comprise at least one metal mixed with the metal-containing corrin compound(s). Such metals are not contained in the corrin structure, but are included in the catalyst in order to enhance the catalytic activity of the corrin compound(s). Appropriate metals include, for example, gold, silver, copper, platinum, palladium, ruthenium, iron, cobalt, and nickel. The ratio between metal(s) and metal-containing corrin compound(s) is not critical because both the metal and corrin-containing compound will provide catalytic activity for oxygen reduction. The metal may be mixed with the metal-containing corrin compound(s) by a physical method, such as strong stirring of the metal and metal-containing corrin compound in an organic solution, followed by drying to yield a catalyst. Alternatively, a chemical method may be utilized. For example, soluble metal salts, such as H₂PtCl₆ (if platinum is the desired metal) may be mixed with metal-containing corrin compound(s) with strong stirring in an organic solution. Subsequently, the mixture may be introduced into a reducing environment, such as one utilizing H₂ as the reducing agent, resulting in the reduction of metal salts to metal. Finally the mixture is preferably dried to yield a catalyst. Other methods that are known or to be discovered for combining a metal with a corrin compound would also be within the scope of the invention.

The invention also relates to a method for preparing a fuel cell cathode catalyst which comprises depositing at least one metal-containing corrin compound, as previously described, onto a conductive support, such as a carbon- or metal-based material, as previously described. The method of deposition of the catalyst onto the conductive support to enhance electrical conductivity is not critical, and may be any method of deposition that is known or to be discovered, including the methods described previously. In one embodiment, the method involves chemisorbing the metal-containing corrin compound from a solution containing an organic or polymeric compound (functioning as a precursor) as previously described, onto the conductive support. Such cathodes may be used to form fuel cells using known methods of fabrication in accordance with the present disclosure.

Finally, the present invention relates to a fuel cell comprising a fuel cell cathode catalyst comprising at least one metal-containing corrin compound, as previously described. Other components of the fuel cell are not critical and are well known in the art, and need not be described.

The invention will now be described in connection with the following, non-limiting example.

### Example 1

### Preparation of Vitamin B12-Derived Catalyst

A sample of vitamin B12 was heated at 600-900° C. in an inert atmosphere to remove the hydrophilic groups and yield a catalyst. The structure of the novel vitamin B12-derived catalyst is not known, but is known to contain a corrin ring, a cobalt metal center, and a cyano group on cobalt. After the heat treatment, the vitamin B12 became insoluble and thus suitable for fuel cell utilization.

### Example 2

### Deposition of Catalyst onto Carbon Black

The catalyst prepared in Example 1 (B12) and carbon black (CB, Vulcan XC-72R) were strongly stirred in a water solution to yield a B12/CB suspension. The suspension was subsequently filtered using filter paper to produce a B12/CB slurry in which the B12 was adsorbed onto the carbon black. The B12/CB slurry was then pyrolyzed in a nitrogen-atmosphere at 600° C. The B12 was then bonded to the carbon black via chemical bonds rather than adsorption, and was thus referred to as “B12 deposited on a conductive support.”

### Example 3

### Deposition of Catalyst onto Electrode and Determination of Catalytic Activity

The vitamin B12-derived catalyst prepared in Example 1 on a conductive support was deposited on the imbedded glassy carbon of a rotating disk electrode (RDE) for catalyzing oxygen reduction, commercially available from PINE Research Instrumentation (RDE type no.: AFE3T050GC).

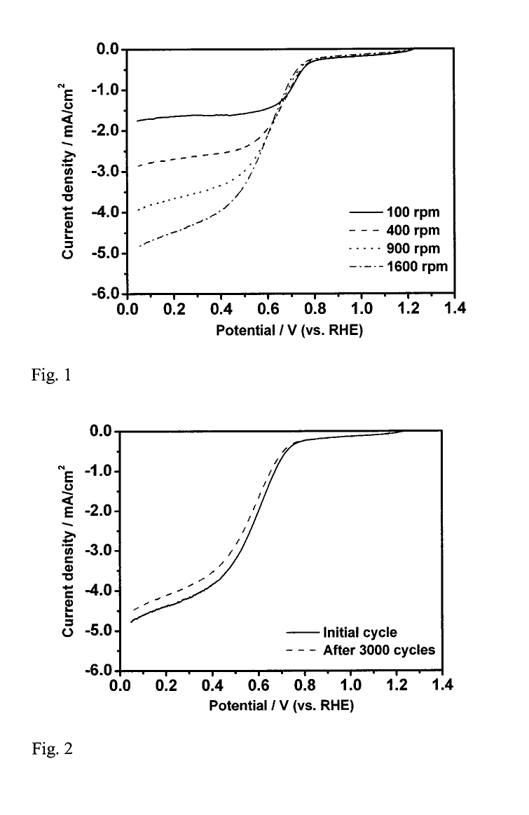

Linear sweep voltammetry (LSV) is an electrochemical methodology to understand the electrochemical behaviors of electrodes or catalysts, in which an electrochemical current is produced by a potential sweep with a fixed scan rate in a desired potential range. In this case, the LSV measurements were conducted in a three-electrode test cell at room temperature using a Solartron Electrochemical System (SI 1280Z). The rotating disk electrode (RDE) was a working electrode. A saturated calomel electrode (SCE) (0.244 V versus reversible hydrogen electrode (RHE)) was a reference electrode and a platinum foil was a counter electrode. The reported potential is versus RHE. For examining catalytic activity of oxygen reduction, the three-electrode cell was tested in an oxygen-saturated 0.5 M H₂SO₄ solution. Specifically, linear sweep voltammetries at various rotating speeds of RDE (100, 400, 900 and 1600 rpm) were measured in oxygen-saturated 0.5 M H₂SO_{4(aq)} at room temperature and the results are shown in FIG. 1.

It can be seen that the vitamin B12-derived catalyst exhibited clear catalytic activity for oxygen reduction with an electron-transfer number of 3.4, indicating that the oxygen reduction reaction of the vitamin B12-derived catalyst preferred the 4 electron-transfer pathway to the 2 electron-transfer pathway. When using LSV measurements by RDE, the current equation is: I⁻¹=Iₖ⁻¹+I_{d}⁻¹, where Iₖ and I_{d} are the kinetic current and the Levich current, respectively. Iₖ can be expressed as: Iₖ=nFAkCΓ and I_{d} can be expressed as: I_{d}=0.201 nFACD^{2/3}v^{1/60}ω^{1/2}, where n is the overall electron-transfer number, F is the Faradic constant, A is the electrode area, k is the kinetic coefficient, C is the concentration of dissolved oxygen, Γ is the surface concentration of the catalyst, D is the diffusion coefficient of oxygen, v is the viscosity of solution, and ω is the rotation speed (unit: rpm) of RDE. Therefore, a Koutecky-Levich plot expressed by I⁻¹ versus ω^{-1/2} can determine the electron-transfer number, n, from the curve slope, 0.201 nFACD^{2/3}v^{1/6}, where F, A, C (1.1×10⁻⁶ mol cm⁻³), D (1.4×10⁻⁵ cm² s⁻¹), and v (10⁻² cm² s⁻¹) are known.

### Example 4

### Determination of Lifetime Cycles

The lifetime cycles of the vitamin B12-derived catalyst in the oxygen-saturated 0.5 M H₂SO_{4(aq)} at room temperature at a rotating speed of 1600 rpm were also measured and the results are shown in FIG. 2. The lifetime cycles were determined by followed Zhang's method (Science, 315(5809); 220-222 (2007)). The curves of initial cycle and after 3000 cycles were recorded at 1600 rpm with a sweep of 10 mV s⁻¹ in the 0.5 M H₂SO₄ solution, as shown in FIG. 2. It can be seen that little degradation was observed for the vitamin B12-derived catalysts after 3000 cycles, demonstrating high stability of the catalyst.

Although such properties are only demonstrated for the vitamin B12-derived catalyst in these Examples, other corrin-based catalysts according to the invention can be prepared and tested according to the present disclosure and would be expected to exhibit similar catalytic activity and stability.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A fuel cell cathode catalyst, wherein the catalyst comprises at least one metal-containing corrin compound.

2. The catalyst according to claim 1, wherein the catalyst further comprises at least one conductive support, and wherein the at least one metal-containing corrin compound is deposited on the at least one conductive support.

3. The catalyst according to claim 2, wherein the at least one conductive support comprises surface functional groups, and wherein the at least one metal-containing corrin compound is covalently attached to the at least one conductive support using a coupling agent bonded to the surface functional groups.

4. The catalyst according to claim 2, wherein the at least one conductive support is selected from the group consisting of a metal-based compound and a carbon-based compound.

5. The catalyst according to claim 1, wherein the catalyst further comprises at least one metal, and wherein the at least one metal-containing corrin compound is mixed with the at least one metal.

6. The catalyst according to claim 5, wherein the at least one metal is selected from the group consisting of gold, silver, copper, platinum, palladium, ruthenium, rhodium, iron, cobalt, and nickel.

7. The catalyst according to claim 1, wherein the at least one metal-containing corrin compound comprises a metal selected from the group consisting of Group 2 metals and transition metals.

8. The catalyst according to claim 7, wherein the at least one metal-containing corrin compound comprises a transition metal selected from Groups 5 to 12 of the periodic table.

9. The catalyst according to claim 8, wherein the at least one metal-containing corrin compound comprises a metal selected from the group consisting of iron, cobalt, and nickel.

10. The catalyst according to claim 8, wherein the at least one transition metal is a reduced type metal or an oxidized type metal.

11. The catalyst according to claim 1, wherein the at least one metal-containing corrin compound comprises a metal complex derived from at least one organic or polymeric compound.

12. The catalyst according to claim 11, wherein the catalyst further comprises at least one conductive support, and wherein the at least one metal-containing corrin compound is chemisorbed from a solution of the at least one organic or polymeric compound onto the at least one conductive support.

13. The catalyst according to claim 1, wherein the at least one metal-containing corrin compound comprises a compound having a formula selected from the group consisting of formulas (3a) to (3d): 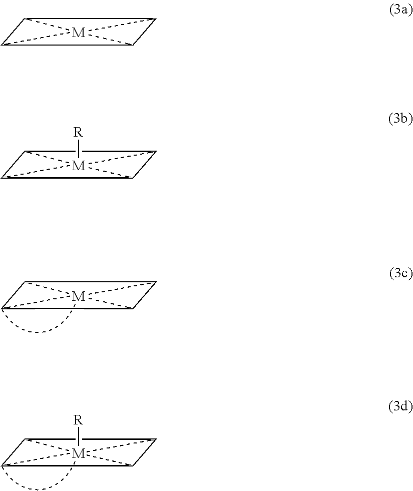
wherein 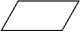
represents a corrin ring, M is a Group 2 metal or a transition metal, and R may be —CN, —OH, or —CₓH_{y}O_{z}Nₘ, wherein x, y, z, and m may independently be 0 or an integer.

14. The catalyst according to claim 13, wherein the at least one metal-containing corrin compound is derived from cyanocobalamin having formula (2):

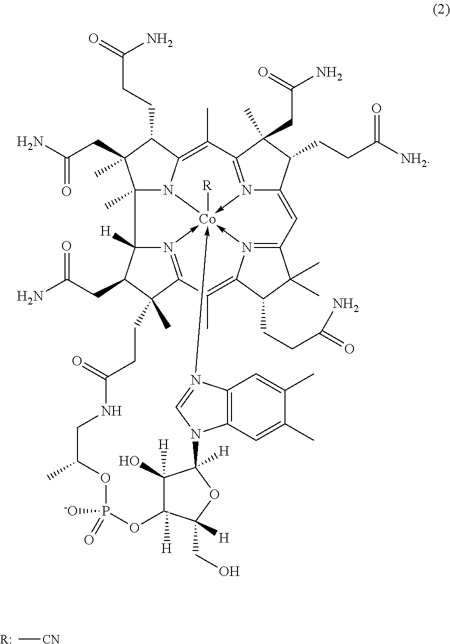

15. A method for forming a fuel cell cathode catalyst, the method comprising depositing at least one metal-containing corrin compound onto a conductive support.

16. The method according to claim 15, wherein the at least one metal-containing corrin compound comprises a metal selected from the group consisting of Group 2 metals and transition metals.

17. The method according to claim 15, comprising chemisorbing the at least one metal-containing corrin compound onto the conductive support from a solution comprising at least one organic or polymeric compound.

18. The method according to claim 15, further comprising mixing at least one metal with the at least one metal-containing corrin compound prior to deposition onto the conductive support.

19. The method according to claim 15, wherein the at least one metal-containing corrin compound is derived from cyanocobalamin having formula (2):

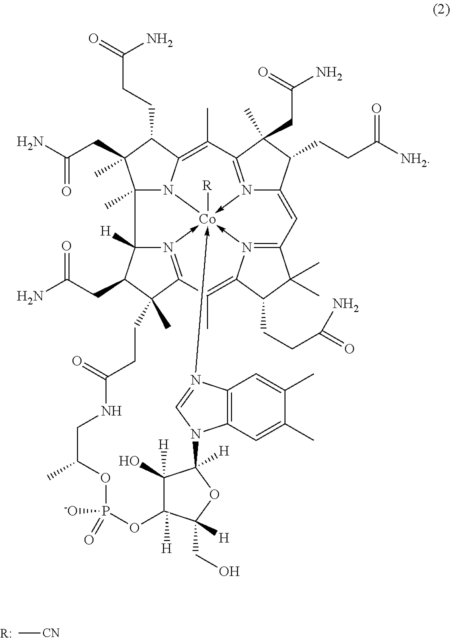

20. A fuel cell comprising a cathode catalyst comprising at least one metal-containing corrin compound.
